(19) United States
(12) Patent Application Publication
(10) Pub. No.: US 2023/0277708 A1
(43) Pub. Date: Sep. 7, 2023

(54) INFORMATION ACQUISITION SYSTEM AND INFORMATION ENCODING ARTICLE

(71) Applicant: CANON KABUSHIKI KAISHA, Tokyo (JP)

(72) Inventors: HIROSHI SAITO, Kanagawa (JP); TAKAHISA ONO, Kanagawa (JP); YUICHIRO MIYAUCHI, Kanagawa (JP); TAKESHI KONDOH, Tokyo (JP); MASARU SUGITA, Tokyo (JP); MASANORI SEKI, Kanagawa (JP); RYUICHI OTSU, Tokyo (JP)

(21) Appl. No.: 18/176,952

(22) Filed: Mar. 1, 2023

(30) Foreign Application Priority Data
Mar. 4, 2022 (JP) 2022-033443
Feb. 3, 2023 (JP) 2023-015333

(51) Int. Cl. A61L 2/28; G01N 21/78; G01N 31/22; G06K 7/14

(52) U.S. Cl. CPC A61L 2/28; G01N 21/78; G01N 31/226; G06K 7/1417

(57) ABSTRACT

An information acquisition system of acquiring information regarding disinfection includes a detection unit configured to detect a variable information code in which a pattern before application of a disinfectant represents a first information code and a pattern after the application of the disinfectant represents a second information code different from the first information code, one or more memories, and one or more processors in communication with the detection unit and with the one or more memories, wherein the one or more processors and the one or more memories are configured to acquire information regarding the disinfection in response to detection of at least the second information code by the detection unit.

### BACKGROUND

### Field of the Disclosure

The present disclosure relates to an information acquisition system and an encoding article.

### Description of the Related Art

With importance paid to operation of cleaning and disinfection, techniques for visualizing the completion of application of a disinfectant are disclosed. In the specification of U.S. Pat. No. 8,519,360, a disinfection operation in the cleaning is visualized by using a fluorescent material in an ultraviolet range. The specification of U.S. Patent Application Publication No. 2017/0336373 discloses a technique of changing the developed color of a pH responsive dye by utilizing a reaction between a disinfectant and the pH responsive dye.

Those specifications of U.S. Pat. No. 8,519,360 and U.S. Patent Application Publication No. 2017/0336373 disclose materials capable of visualizing the completion of the application of the disinfectant, but do not disclose how to record or manage application situations of the disinfectant when there are many disinfection targets. If a person records the completion of the application of the disinfectant by handwriting or by inputting data into, for example, a computer when there are many disinfection targets, such work needs time and effort and leads to a possibility that the application situations of the disinfectant may be erroneously recorded.

### SUMMARY

As a technique of visualizing the completion of the application of the disinfectant, an encoding article on which an information code appears with a change in color developing state caused upon the application of the disinfectant can be used. Such a technique is based on consideration that using a system capable of determining the completion of the application of the disinfectant by reading the information code can reduce errors in comparison with the case of manually recording the data.

However, the above-mentioned technique accompanies a possibility of fraudulent use because the information code can be copied by a camera, a copying machine, or the like.

The present disclosure provides a system for reducing the fraudulent use in the technique of visualizing the completion of the application of the disinfectant with use of the information code.

The present disclosure provides an information acquisition system of acquiring information regarding disinfection, the information acquisition system including a detection unit configured to detect a variable information code in which a pattern before application of a disinfectant represents a first information code and a pattern after the application of the disinfectant represents a second information code different from the first information code, one or more memories, and one or more processors in communication with the detection unit and with the one or more memories, wherein the one or more processors and the one or more memories are configured to acquire information regarding the disinfection in response to detection of at least the second information code by the detection unit.

The present disclosure provides an article including a base, and a color developing portion applied to the base, wherein the color developing portion includes a visualization agent, wherein a color developing state of the visualization agent changes upon application of a disinfectant, wherein the color developing portion forms, on a surface of the base, a variable information code capable of being detected by a detection unit, wherein, before application of a disinfectant, the variable information code is a first information code, and wherein application of the disinfectant changes the color developing state of the visualization agent such that the variable information code changes to a second information code.

According to another aspect, the present disclosure provides an information acquisition system of acquiring information regarding a chemical reaction, the information acquisition system including a detection unit configured to detect a variable information code in which a pattern before occurrence of the chemical reaction represents a first information code and a pattern after the occurrence of the chemical reaction represents a second information code different from the first information code, one or more memories, and one or more processors in communication with the detection unit and with the one or more memories, wherein the one or more processors and the one or more memories are configured to acquire the information regarding the chemical reaction with detection of at least the second information code by the detection unit.

According to another aspect, the present disclosure provides an object including a base and a color developing portion applied to the base, wherein the color developing portion includes a visualization agent, wherein a color developing state of the visualization agent changes upon a chemical reaction, wherein the color developing portion forms, on a surface of the base, a variable information code capable of being detected by a detection unit, wherein, before an occurrence of the chemical reaction, the variable information code is a first information code, and wherein the occurrence of the chemical reaction changes the color developing state of the visualization agent such that the variable information code changes to a second information code.

The present disclosure further provides a method of acquiring information regarding disinfection, the method including detecting a variable information code in which a pattern before application of a disinfectant represents a first information code and a pattern after the application of the disinfectant represents a second information code different from the first information code, and acquiring the information regarding the disinfection with detection of at least the second information code by the detection unit.

Further features of various embodiments will become apparent from the following description of exemplary embodiments with reference to the attached drawings.

### BRIEF DESCRIPTION OF THE DRAWINGS

FIG. 1 is a block diagram to explain a system according to a first embodiment of the present disclosure.

FIG. 2 is a flowchart to explain an example of a method according to the first embodiment of the present disclosure.

FIG. 3 is a flowchart to explain another example of the method according to the first embodiment of the present disclosure.

FIG. 4 is a flowchart to explain still another example of the method according to the first embodiment of the present disclosure.

FIG. 5 illustrates an example of a server device according to the first embodiment of the present disclosure.

FIG. 6 illustrates an example of an encoding article according to a second embodiment of the present disclosure.

FIGS. 7A, 7B, 7C, and 7D are explanatory views of an infectant visualizing sheet that is an example of the encoding article according to the second embodiment of the present disclosure.

### DESCRIPTION OF THE EMBODIMENTS

Embodiments of the present disclosure will be described below, but the present disclosure is not limited to the following embodiments.

### First Embodiment Information Acquisition System

An information acquisition system 100 according to a first embodiment acquires information regarding disinfection. The information acquisition system 100 includes a detection unit 101 configured to detect a variable information code in which a pattern before application of a disinfectant represents a first information code and a pattern after the application of the disinfectant represents a second information code different from the first information code. The information acquisition system 100 further includes an acquisition unit 105 configured to acquire the information regarding the disinfection with detection of the second information code (reading of the second information code) by the detection unit 101.

When, as in this embodiment, the information regarding the disinfection is acquired with the detection of the variable information code indicating the second information code different from the first information code, fraudulent use can be reduced. This is because the second information code appears after the application of the disinfectant and the information regarding the disinfection cannot be obtained without applying the disinfectant. The variable information code is typically a pattern (printed pattern) represented on a base (substrate), such as a sheet of paper or coated paper, and details of the variable information code will be described later.

### Information Regarding Disinfection

The information regarding disinfection in this embodiment is information related to at least any of, for example, whether the disinfectant has been applied, time when the application of the disinfectant has been performed (or determined), and a place where the application of the disinfectant has been performed.

The variable information code may include a region where the pattern changes with the application of the disinfectant and a region where the pattern does not change with the application of the disinfectant.

### Acquisition Unit

The acquisition unit 105 in this embodiment acquires the information regarding the disinfection with the detection unit 101 reading at least the second information code. Thus, the information regarding the disinfection cannot be acquired only with the first information code, and this feature can reduce a possibility of such fraud that, although the disinfectant is not applied in fact, it is determined as if the disinfectant has been applied.

The acquisition unit 105 may acquire the information regarding the disinfection by further using the information regarding the place where the disinfectant has been applied (namely, a disinfected region). The information regarding the place where the disinfectant has been applied can be given by using, for example, GPS (Global Positioning System). Here, the information regarding the place where the disinfectant has been applied may be information regarding a place where a person who has applied the disinfectant resides.

The variable information code may cause at least one of disappearance of part of the pattern forming the first information code upon the application of the disinfectant and appearance of a pattern upon the application of the disinfectant, the pattern being not included in the pattern forming the first information code.

The acquisition unit 105 may acquire the information regarding the disinfection with the detection unit 101 reading both the first information code and the second information code.

The variable information code may indicate the second information code after the application of the disinfectant and thereafter indicate the first information code again.

The variable information code may present a pattern after the application of the disinfectant, the pattern representing a third information code different from the first information code, and the second information code and the third information code may appear at different times after the application of the disinfectant. In this case, the acquisition unit 105 can be configured to acquire the information regarding the disinfection with the detection unit 101 reading the first information code, the second information code, and the third information code.

### Storage Unit

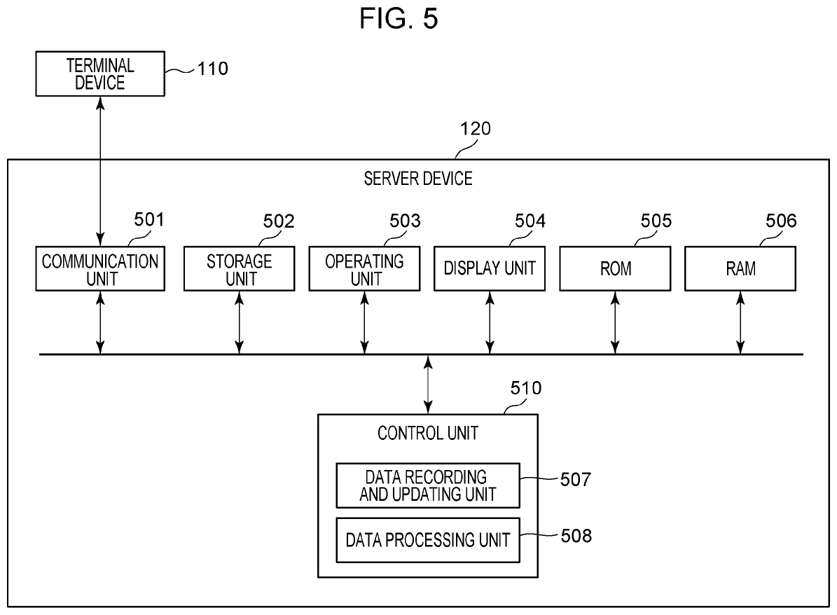

A storage unit (e.g., the storage unit 502 in FIG. 5) in this embodiment stores, in a linked relation, a determination result indicating that the application of the disinfectant has been performed in the disinfected region, the information regarding the place of the disinfected region, and the information regarding the time when the application of the disinfectant has been performed. Since not only the information regarding whether the application of the disinfectant has been performed, but also the information regarding when and where the application of the disinfectant has been performed can be obtained, the disinfection can be efficiently performed in many disinfected regions.

The storage unit in this embodiment may be a memory such as a ROM or a RAM, or a storage medium such as a flexible disk, a hard disk, an optical disk, an opto-magnetic disk, a CD-ROM, a CD-R, a magnetic tape, a nonvolatile memory card, or a DVD.

### Disinfectant

The disinfectant used in this embodiment is not limited to a particular one, but it is typically a liquid composition sprayed to the disinfected region. Effective components of the liquid composition may be, for example, a surfactant, a basic component, an acidic component, and alcohol. Examples of the basic component may be alkali, perchloric acid, hypochlorous acid, and sodium salts of the formers.

### Detection Unit

The information acquisition system 100 according to this embodiment may include a detection unit 101 that is configured to detect an environmental change in the disinfected region and to output information regarding the environmental change. In this case, the disinfection information acquisition unit 105 can determine, based on the information regarding the environmental change output from the detection unit 101, whether the application of the disinfectant has been performed in the disinfected region.

In an example, the information regarding the environmental change in the disinfected region, which is output from the detection unit 101, is output as a numerical value and is compared with a predetermined threshold. Then, if the numerical value is greater than the predetermined threshold, it can be determined that the application of the disinfectant has been performed. When the numerical value is smaller than the predetermined threshold, it can be determined that the application of the disinfectant has not been performed. The threshold can be set as appropriate depending on the disinfected region. The wording “environmental change in the disinfected region” indicates an environmental change in the disinfected region and/or the surroundings of the disinfected region. The surroundings of the disinfected region may cover a range within which the detection unit 101 can detect the environmental change.

The detection unit 101 in this embodiment is not limited to a particular one insofar as it can detect the environmental change in the disinfected region. At least one selected from the group consisting of a unit of detecting an optical change, a unit of detecting vibration, a unit of detecting an electrical change, and a unit of detecting a temperature change can be used as the detection unit 101.

### Unit of Detecting Optical Change

The unit of detecting the optical change in this embodiment detects a change in light obtained from the disinfected region. Here, the change in light indicates at least one of a change in light intensity and a change in light wavelength.

In this embodiment, the unit of detecting the optical change may be, for example, an image capturing unit such as a camera, a video camera, a monitor camera, a terminal, a smartphone, or a tablet, each of the last three having the camera function. A camera (wearable camera or mobile camera) worn by a worker who performs the application of the disinfectant can also be used. A typical example of the unit of detecting the optical change is an image information acquisition unit that may capture a still image or a moving image.

When the image information acquisition unit 105 acquires a moving image, it may include a unit of acquiring an image of the disinfected region from the moving image captured during a certain period. To acquire the image of the disinfected region from the moving image, the image information acquisition unit 105 may include a unit of detecting a changing point in the moving image. In that case, from the viewpoint of security, it is preferable not to store images other than the image of the disinfected region.

The unit of detecting the optical change may detect an optical change in the disinfected region without capturing any image. When the disinfectant is a liquid, for example, whether the liquid has been applied may be detected by using a liquid intrusion indicator. In the case of using a disinfectant visualization sheet described later, for example, the unit may be configured to detect a developed color (particular wavelength) of a visualization agent disposed in the disinfected region. In another example, when the visualization agent is disposed in the form of a two-dimensional code as described later or is arranged to represent character information or image information (figure), the unit may be configured to detect the two-dimensional code, the character information, or the image information.

### Unit of Detecting Vibration

The unit of detecting the vibration in this embodiment may be a unit capable of detecting vibration caused upon the application of the disinfectant, for example, vibration of furniture such as a desk or a chair. The vibration at such a level that can be regarded as indicating the application of the disinfectant may be detected by using, for example, at least one of an acceleration sensor and a speed sensor. The acceleration sensor may be contact type or non-contact type. In the case of the contact type, a piezoelectric, conductive, or strain-gauge sensor can be used. In the case of the non-contact type, a displacement detection sensor of eddy current type, electrostatic capacitance type, or optical type can be used.

### Unit of Detecting Electrical Change

The unit of detecting the electrical change in this embodiment detects a target that causes an electrical change, such as a change in current or voltage, upon the application of the disinfectant. An example of the unit of detecting the electrical change may be a circuit that causes a change in current or voltage upon application of a liquid, such as a pH meter or an alcohol sensor, for example.

### Unit of Detecting Temperature Change

The unit of detecting the temperature change in this embodiment detects a temperature change in the disinfected region and/or the surroundings of the disinfected region. The unit may detect, for example, a temperature drop that is caused due to evaporation heat absorbed after the application of the disinfectant, such as alcohol, to the disinfected region, or a temperature rise of a disinfection target when a person is wiping off the disinfectant applied to the disinfection target. A known temperature sensor can be used as the unit of detecting the temperature change in this embodiment.

### Terminal Communication Unit and Server Communication Unit

A terminal communication unit 102 and a server communication unit 103 in this embodiment communicate information (data) such as the determination result and so on. Those units may be each, for example, a LAN (Local Area Network) or a WAN (Wide Area Network). The LAN may be a wired LAN or a wireless LAN. The terminal communication unit 102 is implemented as, for example, a wireless communication module and executes communication with the server communication unit 103 or a communication network. The above point is similarly applied to the server communication unit 103 as well.

### Terminal Device

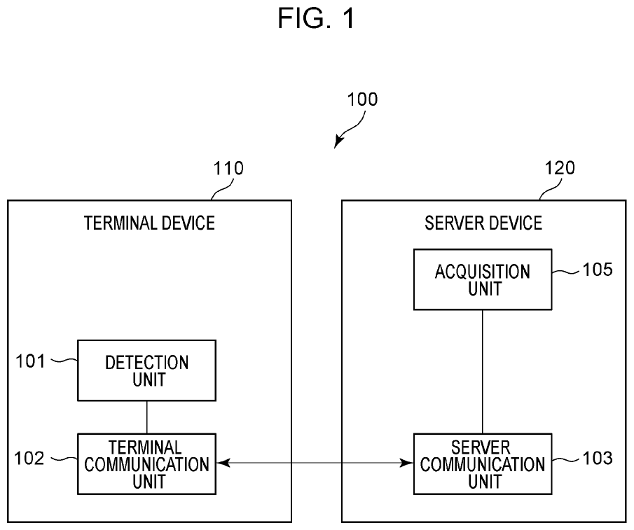

An example of a terminal device 110 in this embodiment includes a detection unit 101, a determination unit, and a terminal communication unit 102 (FIG. 1). The terminal device 110 in this embodiment transmits, at least when the application of the disinfectant is performed, a result of determination as to the application of the disinfectant to a server device 120.

The terminal device 110 in this embodiment may be a terminal dedicated for determining the application of the disinfectant or a universal smartphone or tablet.

### Server Device

The server device 120 in this embodiment has the function of not only communicating information (data) with the terminal device 110 in the disinfected region, but also storing and analyzing the information.

An example of the server device 120 is described with reference to a block diagram of FIG. 5. The server device 120 includes a communication unit 501 (server communication unit 103), a storage unit 502, an operating unit 503, a display unit 504, a ROM 505, a RAM 506, and a control unit 510. The communication unit 501 is implemented as a LAN card or the like and executes communication with an external device (for example, the terminal device 110) or a communication network. The ROM 505 is implemented as a nonvolatile memory or the like and stores various programs and so on. The RAM 506 is implemented as a volatile memory or the like and temporarily stores various kinds of information. The storage unit 502 is implemented as an HDD (Hard Disk Drive), an SSD (Solid State Drive), a NAS (Network Attached Storage), or the like and stores various kinds of information. The operating unit 503 is implemented as a keyboard, a mouse, or the like. The display unit 504 is implemented as a display or the like and displays various kinds of information to a user.

The control unit 510 includes a data recording and updating unit 507 and a data processing unit 508. The data recording and updating unit 507 stores, in the storage unit 502, the ROM 505, or the RAM 506, the information regarding the determination result indicating that the application of the disinfectant (namely, operation of the disinfection) has been performed, and regarding the time and the place at which the application of the disinfectant has been performed. The data processing unit 508 executes data processing of the information by, for example, adding information to the information having been read or converting the read information while referring to a database stored in the storage unit 502, the ROM 505, or the RAM 506. A processing result may be communicated to the terminal device 110 by using the communication unit 501.

### Method

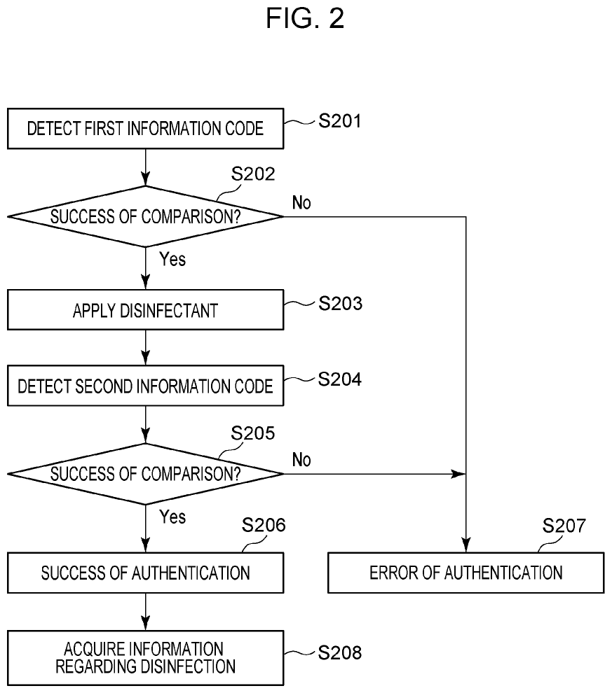

An information acquisition method according to an embodiment of the present disclosure is a method of acquiring the information regarding the disinfection and includes at least the following steps.
(1) A detection step of detecting a variable information code in which a pattern before the application of the disinfectant represents a first information code and a pattern after the application of the disinfectant represents a second information code different from the first information code.
(2) An acquisition step of acquiring the information regarding the disinfection with detection of at least the second information code by the detection unit 101. Examples of the information acquisition method according to the embodiment will be described below with reference to FIGS. 2, 3, and 4.

In the example illustrated in FIG. 2, the first information code is detected (S201) and subjected to comparison (S202). If the comparison succeeds, the disinfectant is applied (S203). When the second information code appears with the application of the disinfectant, the second information code is detected by the detection unit 101 (S204) and is subjected to comparison (S205). If the comparison succeeds, success of authentication is determined (S206). If the comparison does not succeed in S202 or S205, error of the authentication is determined (S207). If the authentication succeeds, the information regarding the disinfection is acquired (S208).

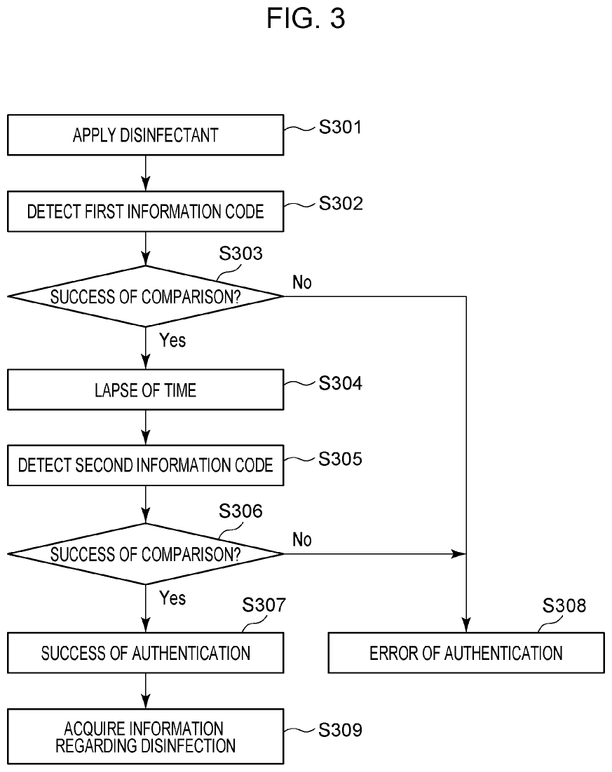

FIG. 3 illustrates the example in which the disinfection is performed at earlier timing while a reversible encoding article is used.

The variable information code used in the example illustrated in FIG. 3 changes from the first information code to the second information code after the lapse of a certain time from the application of the disinfectant.

In this example, the disinfectant is applied (S301). Before the pattern represented by the variable information code changes from the first information code to the second information code, the first information code is detected by the detection unit 101 (S302) and is subjected to comparison (S303). If the comparison succeeds and then a certain time lapses (S304), the second information code appears. The second information code having appeared is detected (S305) and subjected to comparison (S306). If the comparison succeeds, success of authentication is determined (S307). If the comparison does not succeed in S303 or S306, error of the authentication is determined (S308). If the authentication succeeds, the information regarding the disinfection is acquired (S309).

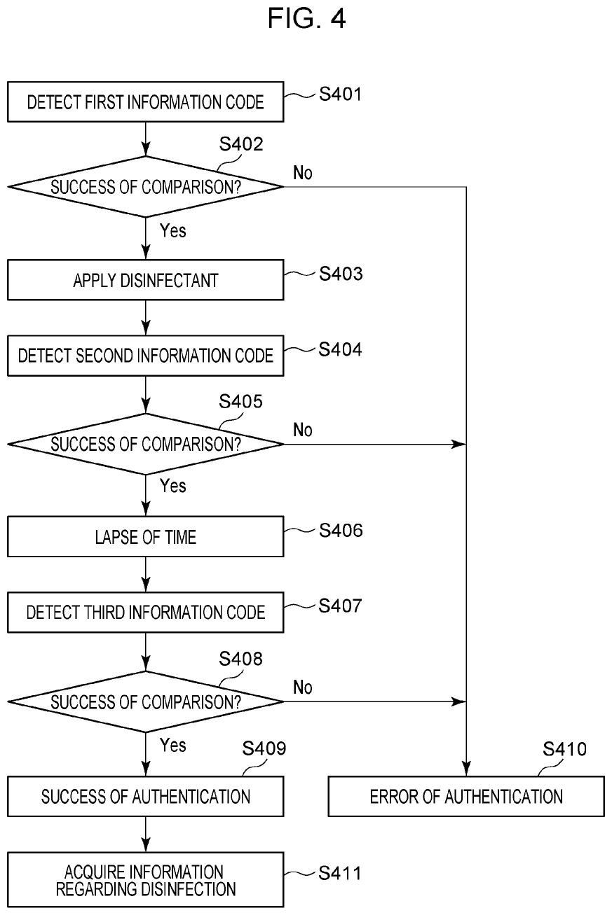

In the example illustrated in FIG. 4, the variable information code variably represents three different information codes. It is to be noted that a variable information code variably representing four or more different information codes may also be used in the information acquisition method according to the embodiment.

In this example, the first information code is detected (S401) and subjected to comparison (S402). If the comparison succeeds, the disinfectant is applied (S403). When the second information code appears with the application of the disinfectant, the second information code is detected by the detection unit 101 (S404) and is subjected to comparison (S405). If the comparison succeeds and then a certain time lapses (S406), a third information code appears. The third information code having appeared is detected (S407) and subjected to comparison (S408). If the comparison succeeds, success of authentication is determined (S409). If the comparison does not succeed in S402, S405, or S408, error of the authentication is determined (S410). If the authentication succeeds, the information regarding the disinfection is acquired (S411).

An example of the information acquisition system 100 according to the embodiment will be described below with reference to FIG. 1. The embodiment is described in connection with an example in which the detection unit 101 is a monitor camera capable of detecting the environmental change in the disinfected region.

In this example, a camera is used as the detection unit 101 to detect the environmental change in the disinfected region, namely the application of the disinfectant. In such a case, when an image specific to the application of the disinfectant is acquired, it is determined that the application of the disinfectant has been performed. For instance, when it is determined that a disinfection worker is making an action to wipe off the disinfectant, this is determined as indicating that the application of the disinfectant has been performed. The determination result indicating that the application of the disinfectant has been performed is transmitted to the server device 120 together with the information regarding the time of the determination that the application of the disinfectant has been performed and the place where the application of the disinfectant has been performed. The storage unit 502 in the server device 120 stores, in a linked relation, plural items of information regarding the above-described determination result, the time when the application of the disinfectant has been performed (determined), and the place where the application of the disinfectant has been performed.

The above-described plural items of information are similarly stored in the storage unit 502 for each of many disinfected regions.

An analysis unit provides, based on the information stored in the storage unit 502, information (recommended information) indicating in which order the disinfection is to be performed for the disinfected regions.

According to this embodiment, since the monitor cameral is used, there is no necessity of disposing a new device to collect the information for determining the application of the disinfectant.

### Second Embodiment Encoding Article

An encoding article according to a second embodiment is described. The encoding article according to this embodiment includes a variable information code capable of being read by a detection unit 101. The encoding article has an encoding region where the variable information code is formed, and represents, in the encoding region, a first information code before the application of the disinfectant and a second information code after the application of the disinfectant.

The configuration of the variable information code described above in the first embodiment can be applied to the encoding article according to this embodiment.

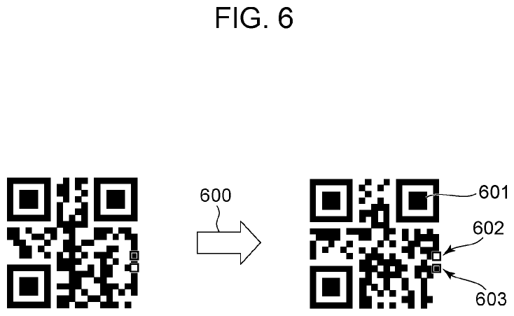

FIG. 6 illustrates an example of the encoding article. Reference numeral 601 denotes an ordinary printed region where no change occurs, 602 denotes a color disappearing portion, and 603 denotes a color developing portion. A figure on a left side in FIG. 6 represents the first information code, and a figure on a right side represents the second information code. The information code changes with a chemical reaction, specifically, the application of the disinfectant (denoted by 600).

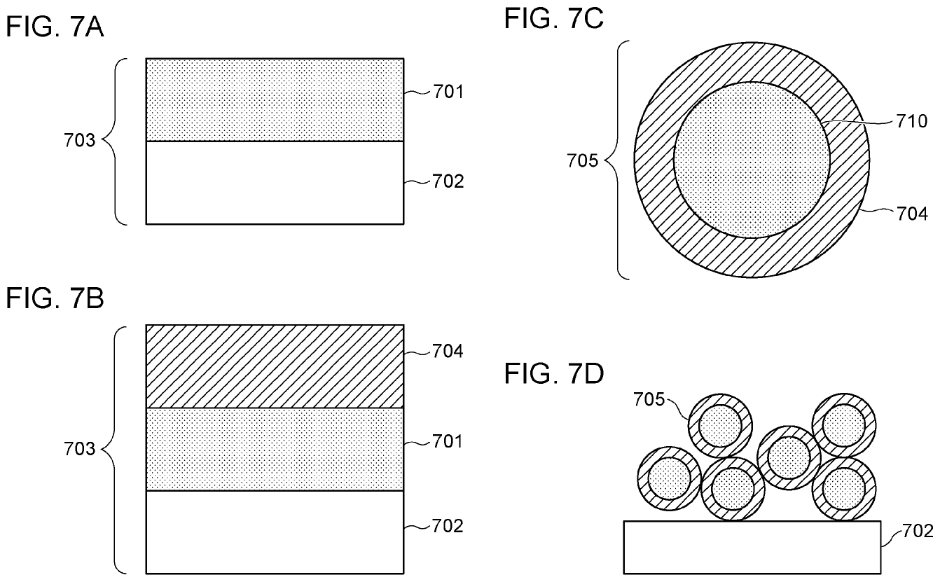

A disinfectant visualization sheet as an example of the encoding article is described, by way of example, with reference to FIGS. 7A, 7B, 7C, and 7D.

### Disinfectant Visualization Sheet

The disinfectant visualization sheet 703 according to this embodiment includes a base 702 and a color developing portion 701 disposed on the base 702 (FIG. 7A). The color developing portion 701 includes the visualization agent (not illustrated) of which color developing state changes with the application of the disinfectant. Using the disinfectant visualization sheet according to this embodiment enables the application of the disinfectant to be simply confirmed by visually checking a change in the color developing state. Furthermore, the disinfectant and the visualization agent are mixed with each other at the time of applying the disinfectant to the disinfectant visualization sheet, and a time during which the disinfectant and the visualization agent are in a mixed state is short. Accordingly, materials used as the disinfectant and the visualization agent are easy to store for a long term.

### Color Developing Portion

The color developing portion in this embodiment includes at least the visualization agent. The color developing portion may include a binder to increase adhesion to the base. Resin can be used as the binder. For example, urethane resin or polyvinyl alcohol can be used.

A thickness of the color developing portion is preferably 1 μm or more and 80 μm or less. The thickness is more preferably 5 μm or more and 60 μm or less and particularly preferably 15 μm or more and 50 μm or less. If the color developing portion is thicker than 80 μm, this is undesirable because the adhesion to the base reduces. If the color developing portion is thinner than 1 μm, this is undesirable because the color is not developed sufficiently.

### Visualization Agent

The visualization agent used in this embodiment is not limited to a particular one insofar as the color developing state of the visualization agent changes upon the reaction with the disinfectant. In this embodiment, the wording “change in the color developing state” indicates such a change as enabling a color difference between before and after the application of the disinfectant to be visually recognized. That change is, for example, any of a change from a visually unrecognizable state to a visually recognizable state, a change from a visually recognizable state to a visually unrecognizable state, and a visually recognizable color change. Here, the visually recognizable color change includes a change in shade of color.

Chromism is known as an example of phenomenon causing the change in the color developing state. The chromism includes, for example, photochromism, thermochromism, electrochromism, acidichromism, solvatochromism, and vapochromism. In this embodiment, substances causing those phenomena are called chromic substances. More specifically, those chromic substances can be called a photochromic substance, a thermochromic substance, an electrochromic substance, an acidichromic substance, a solvatochromic substance, and a vapochromic substance.

The acidichromic substance is a substance of which color changes with a change in pH, and a color developing range is different depending on the properties of individual substances. The acidichromic substance is, for example, at least one selected from the group consisting of Metanil Yellow, Metacresol Purple, Thymol Blue, Tropaeolin O, 2,4-dinitrophenol, Methyl Yellow, Bromophenol Blue, Congo Red, Methyl Orange, Bromochlorophenol Blue, Alizarin Red S, Bromocresol Green, Methyl Orange-Xylene Cyanol FF, 2,5-dinitrophenol, Methyl Orange-Indigo Carmine, Methyl Red, Methyl Orange-Xylene Cyanol FF-phenolphthalein, Lacmoid, Chlorophenol Red, o-nitrophenol, p-nitrophenol, Bromocresol Green-Methyl Red, Bromocresol Purple, Bromophenol Red, Methyl Red-Methylene Blue, Bromothymol Blue, Neutral Red, Phenol Red, Neutral Red-Bromothymol Blue, Cresol Red, α-naphtolphthalein, Bromothymol Blue-Phenol Red, Curcumin, phenolphthalein, Cresol Red-Thymol Blue, o-cresolphthalein, α-naphtolbenzein, thymolphthalein, Thymol Blue-phenolphthalein, Alizarin Yellow GG, Alizarin Yellow R, nitramine, 1,3,5-trinitrobenzene, Indigo Carmine, Methyl Violet, litmus, and Methyl Purple. Colors of some of the above-mentioned examples change in either acidic or alkaline pH range while colors of some other examples, such as Thymol Blue, change in both acidic and alkaline pH ranges. Among the above-mentioned examples, desirable examples because of being low toxic are Metanil Yellow, Metacresol Purple, Thymol Blue, Tropaeolin O, Bromophenol Blue, Bromochlorophenol Blue, Alizarin Red S, Bromocresol Green, Methyl Red, Lacmoid, Chlorophenol Red, o-nitrophenol, Bromocresol Purple, Bromophenol Red, Bromothymol Blue, Neutral Red, Phenol Red, Cresol Red, α-naphtolphthalein, phenolphthalein, o-cresolphthalein, thymolphthalein, Alizarin Yellow GG, Alizarin Yellow R, Methyl Violet, litmus, and Methyl Purple. More desirable examples because of causing a color change at pH of 3 or more and 11 or less are Metacresol Purple, Thymol Blue, Tropaeolin O, Bromophenol Blue, Bromochlorophenol Blue, Alizarin Red S, Bromocresol Green, Methyl Red, Lacmoid, Chlorophenol Red, o-nitrophenol, Bromocresol Purple, Bromophenol Red, Bromothymol Blue, Neutral Red, Phenol Red, Cresol Red, α-naphtolphthalein, phenolphthalein, o-cresolphthalein, thymolphthalein, Alizarin Yellow GG, Alizarin Yellow R, litmus, and Methyl Purple. It is desirable that the color change occurs in an alkaline range when the disinfectant used is alkaline, and in an acidic range when the disinfectant used is acidic.

The color of the visualization agent changes with vaporization of the disinfectant to restore the original one. Here, the word “restore” indicates that the color of the visualization agent returns to the state before the application of the disinfectant. This can also be said that the color developing state is reversible. In other words, the visualization agent used in this embodiment may have the color developing state that changes for a certain time with the application of the disinfectant and then returns to the state before the application of the disinfectant. For example, the color developing state may change to the visually recognizable state with the application of the disinfectant to the visualization agent that is in the visually unrecognizable state, and may return to the visually unrecognizable state after the lapse of a certain time. When the visualization agent is reversible as described above, the visualization agent can be used in a situation in which the disinfection needs to be performed repeatedly.

Two or more types of visualization agents may be used. Using, for example, visualization agents keeping developed colors for different times is desirable in that the visualization agents can serve as indicators indicating the time lapsed after contact with the disinfectant.

The visualization agent may be made of a material responsive to light other than visible light and may be made of a material absorbing light of wavelength in an ultraviolet or infrared range. The ultraviolet or infrared range includes a near-ultraviolet or near-infrared range. In such a case, an observation system for the ultraviolet or infrared range is disposed in the above-described monitor camera. Those materials and observation system can be given by known ones.

### Base

The base used in this embodiment may be any type of member insofar as the color developing portion can be disposed on the base.

The base is preferably a resin sheet because the resin sheet is light and flexible. Materials of the resin sheet include, for example, polyester resin such as polyethylene terephthalate, polybutylene terephthalate, or a polyethylene terephthalate/isophthalate copolymer; polyolefin resin such as polyethylene, polypropylene, or polymethylpentene; polyfluoroethylene resin such as polyvinyl fluoride, polyvinylidene fluoride, polytetrafluoroethylene, or an ethylene-tetrafluoroethylene copolymer; aliphatic polyamide resin such as nylon 6 or nylon 6,6; vinyl polymer resin such as polyvinyl chloride, a vinyl chloride/vinyl acetate copolymer, an ethylene/vinyl acetate copolymer, an ethylene/vinyl alcohol copolymer, polyvinyl alcohol, or vinylon; cellulose resin such as cellulose triacetate or cellophane; acrylic resin such as polymethyl methacrylate, polyethyl methacrylate, polyethyl acrylate, or polybutyl acrylate; and other synthetic resins such as polystyrene, polycarbonate, polyarylate, and polyimide. The resin sheet may be used as one type of sheet, or a composite or laminated sheet formed by two or more types of sheets. The base preferably includes an accepting layer to retain the color developing portion. This is because, when the visualization agent is ink, the accepting layer can easily retain the ink.

The accepting layer of the base in this embodiment is desired not to react with the visualization agent. For instance, when the visualization agent develops the color in the alkaline range, the accepting layer is desirably neutral or acidic. If the accepting layer and the visualization agent react with each other, this is undesirable because the visualization agent develops the color before reacting with the disinfectant.

The base in this embodiment may be transparent, opaque, or colored. When the visualization agent has a color before reacting with the disinfectant, the base is desirably colored in a color close to that of the visualization agent.

Release paper, a metal plate, a piece of wood, or the like can be used as the base in this embodiment. The base may further include a bonding portion for bonding of the base to another member.

### Retaining Portion

The disinfectant visualization sheet 703 in this embodiment may include a retaining portion 704 to retain the disinfectant on the color developing portion 701 (FIG. 7B). Since the retaining portion 704 enables the disinfectant to be retained for a longer time, it is possible to prolong a time during which the disinfectant is reacting with the visualization agent and developing the color. Furthermore, the color developing time can be controlled by appropriately selecting a material used as the retaining portion 704. For example, a time to change from the visually recognizable color-developed state to the visually unrecognizable state can be controlled.

Because of having the above-described function, the retaining portion can also be said as a color-disappearing time control portion.

The retaining portion desirably includes voids to retain the applied disinfectant. For example, inorganic particles or resin having voids can be used to form the voids in the retaining portion. The inorganic particles used here are at least one selected from the group consisting of pearl-necklace silica particles, chain silica particles, spherical colloidal silica particles, aspherical colloidal silica particles, alumina particles, titania particles, and zirconia particles. A peak position of particle size distribution measured for particle sizes of the inorganic particles in accordance with the dynamic light scattering method is preferably present at 1 nm or more and 300 nm or less. The peak position is more preferably present at 1 nm or more and 150 nm or less and is even preferably present at 1 nm or more and 60 nm or less. Particularly, the peak position of the pearl-necklace silica particles is preferably present at 20 nm or more and 50 nm or less. The peak position of the chain silica particles is preferably present at 1 nm or more and 10 nm or less. If the particle size is too large, this is undesirable because the number of voids reduces. On the other hand, if the particle size is too small, this is undesirable because pore diameters of the voids formed by the inorganic particles become too small and an absorption speed of the applied disinfectant reduces. A distribution curve of the pore diameters preferably has a maximum peak in a range of 5 nm or more and 20 nm or less. If the peak of the distribution curve of the pore diameters is less than 5 nm, the absorption speed of the disinfectant may reduce in some cases. If that peak exceeds 20 nm, haze may increase in some cases. An increase of the haze is undesirable in that whiteness becomes conspicuous and visibility of the color developing portion reduces. The pore diameters can be determined with the BET specific area measurement.

The resin used here can be selected from various types of resins. However, when the disinfectant is aqueous, water-soluble resin is preferably used. The water-soluble resin may be, for example, at least one selected from the group consisting of cellulose binders such as methylcellulose, methyl hydroxyethyl cellulose, methyl hydroxypropyl cellulose, and hydroxyethyl cellulose, starch and modified starch, gelatin and modified gelatin, natural polymer resins such as casein, pullulan, Arabic gum, karaya gum, and albumin, including derivatives thereof, polyvinyl alcohol and modified polyvinyl alcohol, latexes and emulsions such as SBR latex, NBR latex, a methyl methacrylate-butadiene copolymer, and an ethylene-vinyl acetate copolymer, polyacrylamide, vinyl polymers such as polyvinyl pyrrolidone, polyethyleneimine, polypropylene glycol, polyethylene glycol, and maleic anhydride, and copolymers thereof.

A thickness of the retaining portion does not need to be uniform. For instance, a retention time of the disinfectant can be controlled by changing the thickness in part of the retaining portion. This is desirable in that the color developing time in the color developing portion can be controlled and the color developing portion can serve as an indicator indicating the time lapsed after contact with the disinfectant.

### Protection Portion

In this embodiment, a protection portion for protecting the color developing portion may be disposed on the color developing portion. Here, the wording “on the color developing portion” indicates not only the case in which the protection portion is disposed in contact with the color developing portion, but also the case in which the protection portion is disposed on the retaining portion when the retaining portion is disposed on the color developing portion. For instance, when the water-soluble resin is used for the retaining portion, a surface of the retaining portion is preferably coated with a protection layer because the surface becomes viscous. The protection portion in this embodiment can be made of at least one selected from the group consisting of resins such as acrylic resin, vinyl acetate resin, vinyl chloride resin, an ethylene/vinyl acetate copolymer resin, polyamide resin, polyester resin, urethane resin, and polyolefin resin, and copolymer resins of the formers.

The provision of the protection portion is further preferable from the viewpoint of giving durability to the color-disappearing time control portion because the surface of the disinfectant visualization sheet is wiped with the disinfectant not a few times.

### Information Code, Two-Dimensional Code

In the disinfectant visualization sheet according to this embodiment, the visualization agent can be disposed such that the information code appears as a pattern. An example of the information code may be a two-dimensional code.

The two-dimensional code is at least one selected from the group consisting of QR code (registered trademark), micro QR code, SP code, VeriCode, MaxiCode, CP code, Data Matrix, DataMatrix ECC200, Code 1, AztecCode, INTACTA CODE, Card e, Chameleon Code, PDF417, micro PDF417, Code49, Code16K, Codablock, SuperCode, Ultra Code, RSS Composite, and AztecMesa. Since the visualization agent is disposed on the base to form the two-dimensional code, it is possible to read the two-dimensional code by the above-described image capturing unit and to determine, based on image information of the read two-dimensional code, that the disinfectant has been applied.

The information regarding the time when the disinfectant has been applied and the information regarding the place where the disinfectant has been applied can be obtained from the two-dimensional code. In response to read of the two-dimensional code, character information or image information indicating the completion of the disinfection (cleaning) may be displayed on a display unit that is in, for example, the terminal having read the two-dimensional code. When the disinfected region is a place where usage fee is to be paid, such as a shop or a coworking space, the terminal may be connected to a system of paying the accompanying usage fee upon the read of the two-dimensional code. In addition, a user can be motivated to the disinfection (cleaning) by indicating, upon the read of the two-dimensional code, information regarding grant or exchange of points available in the shop, etc. and regarding giving of a discount and a gift in the form of character information, for example.

### Other Examples of Disinfectant Visualization Sheet

In another example of the disinfectant visualization sheet according to this embodiment, the color developing portion may include visualization particles each including the visualization agent and a carrier particle that carries the visualization agent. An aggregate of the visualization particles can function as the color developing portion. When the visualization particles are used, positions of a retaining layer and a layer including the visualization particles can be designed in microscopic scale, and hence the disinfectant visualization sheet can be designed with precise characteristics.

### Invisible Portion

In still another example of the disinfectant visualization sheet according to this embodiment, several principles can be used in a combined manner in addition to the above-described visualization agent. An invisible portion may be disposed in the above-described sheet including the visualizable color developing portion. In this case, for example, a material not responsive to the visible light applied to the visualization agent is used in a combined manner. An invisible material absorbing the light of wavelength in the ultraviolet or infrared range may be disposed in the invisible portion in this embodiment. The ultraviolet or infrared range includes the near-ultraviolet or near-infrared range. The provision of the invisible portion enables the application of the disinfectant to be further confirmed from the third person perspective in addition to the visual confirmation.

In still another example of the disinfectant visualization sheet according to this embodiment, the thermochromism can also be used in a combined manner. For example, a dye developing different colors depending on temperature is additionally mixed into the visualizable color developing portion. By using such a dye, the developed color changes depending on not only whether the presence of the disinfectant, but also a change in temperature from that in the usage environment of the disinfectant. Thus, a history of the disinfectant visualization sheet, such as a finger touch to the same, can be indirectly recognized. The thermochromism dye can be selected from known substances.

Among the invisible materials used in this embodiment, the dye absorbing the light in the infrared range can be given as at least one selected from the group consisting of a phthalocyanine dye, a naphthalocyanine dye, a metal complex dye, a polymethine dye, a quinone dye, an azo dye, a diphenylmethane or triphenylmethane dye, a radical dye, a perimidine dye, and an Au nanorod. The dye absorbing the light in the ultraviolet range and emitting fluorescence can be given as at least one selected from the group consisting of merocyanine, perylene, acridine, luciferin, pyranine, stilbene, rhodamine, coumarin, and fluorescein.

### Visible Wavelength Range

The visible wavelength range in this embodiment is a wavelength range of 360 nm or longer and 830 nm or shorter. The infrared wavelength range is 900 nm or longer and 14 μm or shorter, and the ultraviolet wavelength range is 100 nm or longer and 400 nm or shorter.

### Marking Composition

The color developing portion disposed in the disinfectant visualization sheet according to this embodiment may be disposed as a marking indicating whether the application of the disinfectant (the cleaning) has been performed. A material used for the marking in this embodiment can be called a marking composition. The marking composition contains the above-described visualization agent and a solvent. The solvent may be, for example, a hydrophobic solvent or an aqueous solvent. The hydrophobic solvent may be, for example, an organic solvent such as heptane or petroleum ether, and the aqueous solvent may be, for example, water or alcohol.

The aqueous solvent used in this embodiment may be water or a mixed solvent using water as a main solvent and adding a protonic organic solvent or a non-protonic organic solvent. The organic solvent used in this embodiment is preferably mixed with or dissolved in water at any desired ratio and is preferably a homogeneously mixed solvent containing 50% by mass or more of water. Deionized water (ion-exchange water) or ultrapure water is preferably used as the water.

The protonic organic solvent is an organic solvent with a hydrogen atom bonded to oxygen or nitrogen (namely, an acidic hydrogen atom). The non-protonic organic solvent is an organic solvent with no acidic hydrogen atom. Examples of the organic solvent may be alcohols, alkylene glycols, polyalkylene glycols, glycol ethers, glycol ether esters, carboxylic amides, ketones, keto-alcohols, cyclic ethers, and so on.

For example, water, a water/ethanol mixed solvent, a water/ethylene glycol mixed solvent, or a water/N-methyl pyrrolidone mixed solvent may be preferably used as the aqueous solvent. The content of the water is preferably 10.0% by mass or more and 90.0% by mass or less and more preferably 50.0% by mass or more and 90.0% by mass or less on the basis of the total mass of the composition.

The content of the aqueous organic solvent in the composition is preferably 5.0% by mass or more and 90.0% by mass or less and more preferably 10.0% by mass or more and 50.0% by mass or less on the basis of the total mass of the composition.

### Other Additives

The marking composition used in this embodiment may optionally contain, as required, polyalcohols such as trimethylolpropane and trimethylolethane, urine, and other water-soluble organic compounds including urine derivatives such as ethylene urine, in addition to the above-mentioned components. The marking composition used in this embodiment may further contain, as required, various additives such as a surfactant, a pH regulator, an anticorrosive agent, an antiseptic agent, a fungicide, an oxidation inhibitor, a reduction inhibitor, a vaporization accelerator, a chelating agent, and water-soluble resin. Examples of the surfactant may be anionic, cationic, and nonionic surfactants. The content of the surfactant in the marking composition is preferably 0.1% by mass or more and 5.0% by mass or less and more preferably 0.1% by mass or more and 2.0% by mass or less on the basis of the total mass of the composition. Nonionic surfactants, such as polyoxyethylene alkyl ether, polyoxyethylene aliphatic ether, polyoxyethylene alkyl phenyl ether, a polyoxyethylenepolyoxypropylene block copolymer, and acetylene glycol compounds, are preferable examples of the surfactant in practical use.

When an image is recorded on a recording medium by discharging the marking composition in this embodiment from an inkjet recording head, the marking composition is preferably used under proper control of surface tension and viscosity. More specifically, a concentration of the color developing compound in the marking composition is preferably about 5% to 20%. The surface tension of the marking composition at 25° C. is preferably 10 mN/m or more and 60 mN/m or less, more preferably 20 mN/m or more and 60 mN/m or less, and particularly preferably 30 mN/m or more and 50 mN/m or less.

The viscosity of the marking composition at 25° C. is preferably 1.0 mPa·s or more and 10 mPa·s or less and more preferably 1.0 mPa·s or more and 5 mPa·s or less.

### Image Recording Method (Method of Applying Marking Composition)

An image recording method in this embodiment relates to a method of applying the marking composition to the base. Various methods, for example, an inkjet method, a flexographic method, a screen method, an offset method, and a spin coating method, can be optionally used as the image recording method in this embodiment. In an example, the inkjet method is a method of recording an image on the recording medium by discharging the marking composition in this embodiment from the inkjet recording head. A method of discharging the marking composition may be a method of applying dynamic energy to the marking composition or a method of applying thermal energy to the marking composition. A process for the inkjet recording method can be implemented by a known method except for using the marking composition in this embodiment.

### Disinfectant Visualization Particle

The color developing state of a disinfectant visualization particle 705 in this embodiment changes with the application of the disinfectant. In more detail, the disinfectant visualization particle includes a color developing layer including the visualization agent of which color developing state changes with the application of the disinfectant, and a carrier particle 710 carrying the visualization agent (FIG. 7C). The disinfectant visualization particle 705 in this embodiment may include the color-disappearing time control portion (not illustrated). The color-disappearing time control portion is not always required to have a layer form. A porous particle can be used to form the color-disappearing time control portion. For example, at least one selected from the group consisting of a meso-porous silica particle, a porous silica particle, a porous titania particle, a porous zirconia particle, a porous ceria particle, a porous zinc oxide particle, a porous crosslinked methyl polymethacrylate particle, a porous crosslinked polystyrene particle, and a porous methyl methacrylate-styrene copolymer crosslinked particle. The disinfectant visualization particle including the color developing layer can be formed by impregnating the above-mentioned porous particle with a solution in which the visualization agent is dissolved, and then by drying them.

A surface of the porous particle may be treated to be hydrophilic or hydrophobic. When the disinfectant is aqueous, the particle surface is preferably hydrophilic. When the disinfectant is not aqueous, the particle surface is preferably hydrophobic.

The disinfectant visualization particle has high rigidity, and this is preferable in that the disinfectant visualization particle has high durability even when the disinfectant visualization sheet is wiped with the disinfectant not a few times.

A particle size of the disinfectant visualization particle is preferably 30 nm or more and 5 μm or less, more preferably 50 nm or more and 3 μm or less, and particularly preferably 80 nm or more and 1 μm or less. If the particle size is smaller than 30 nm, this is undesirable because dispersion efficiency is poor and stability in storing is problematic. If the particle size is greater than 5 this is undesirable because the disinfectant visualization particle tends to easily precipitate when dispersed into a solution.

### Sheet Using Disinfectant Visualization Particles

The disinfectant visualization particles in this embodiment can be used in the form of a sheet including the disinfectant visualization particles by placing the disinfectant visualization particles 705 on the base 702 as illustrated in FIG. 7D.

### Coating Solution of Disinfectant Visualization Particles and Coating Layer

The disinfectant visualization particles in this embodiment can be used in the form of a coating solution, such as a dispersion solution or a slurry. A coating layer can be formed by applying the coating solution to an article.

### Variation Example of Disinfectant Visualization Particle

The disinfectant visualization particle in this embodiment is not always required to have a particulate shape. The disinfectant visualization particle may be in the form of, for example, a rod, a plate, or a film. When the disinfectant visualization particle is in the form of a film, it is particularly desirable to dispose the color-disappearing time control portion from the viewpoint of maintaining the strength against the wiping-off of the disinfectant.

### OTHER EMBODIMENTS

An information acquisition system 100 according to another embodiment of the present disclosure acquires information regarding a chemical reaction. The information acquisition system 100 includes a detection unit 101 configured to detect a variable information code in which a pattern before the occurrence of the chemical reaction represents a first information code and a pattern after the occurrence of the chemical reaction represents a second information code different from the first information code. The information acquisition system 100 further includes an acquisition unit 105 configured to acquire the information regarding the chemical reaction with the detection unit 101 reading the second information code.

An encoding article according to another embodiment of the present disclosure includes a variable information code capable of being read by a detection unit 101. The encoding article has a encoding region where the variable information code is formed, and represents, in the encoding region, a first information code before the occurrence of the chemical reaction and a second information code after the occurrence of the chemical reaction.

### OTHER EMBODIMENTS

Some embodiment(s) can also be realized by a computer of a system or apparatus that reads out and executes computer-executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer-executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer-executable instructions. The computer-executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)?), a flash memory device, a memory card, and the like.

The information acquisition system according to the present disclosure can provide the system for reducing fraudulent use in the technique of visualizing the operation of the disinfection with use of the information code.

While the present disclosure has described exemplary embodiments, it is to be understood that some embodiments are not limited to the disclosed exemplary embodiments. The scope of the following claims is to be accorded the broadest interpretation so as to encompass all such modifications and equivalent structures and functions.

This application claims priority to Japanese Patent Application No. 2022-033443, which was filed on Mar. 4, 2022, and No. 2023-015333, which was filed on Feb. 3, 2023, and which are hereby incorporated by reference herein in their entirety.

What is claimed is:

1. An information acquisition system of acquiring information regarding disinfection, the information acquisition system comprising:
a detection unit configured to detect a variable information code in which a pattern before application of a disinfectant represents a first information code and a pattern after the application of the disinfectant represents a second information code different from the first information code;
one or more memories; and
one or more processors in communication with the detection unit and with the one or more memories, wherein the one or more processors and the one or more memories are configured to:
acquire information regarding the disinfection in response to detection of at least the second information code by the detection unit.

2. The information acquisition system according to claim 1, wherein the variable information code includes a region where the pattern changes with the application of the disinfectant and a region where the pattern does not change with the application of the disinfectant.

3. The information acquisition system according to claim 1, wherein the information regarding the disinfection includes information regarding a place where the disinfectant has been applied.

4. The information acquisition system according to claim 1, wherein the variable information code causes at least one of disappearance of part of the pattern forming the first information code upon the application of the disinfectant and appearance of a pattern upon the application of the disinfectant, the pattern being not included in the pattern forming the first information code.

5. The information acquisition system according to claim 1, wherein the one or more processors and the one or more memories are further configured to acquires the information regarding the disinfection with the detection unit detecting both the first information code and the second information code.

6. The information acquisition system according to claim 1, wherein the variable information code indicates the second information code after the application of the disinfectant, and
after a period of time, indicates the first information code again.

7. The information acquisition system according to claim 1, wherein the variable information code presents a pattern after the application of the disinfectant, the pattern representing a third information code different from the first information code, and the second information code and the third information code appear at different times after the application of the disinfectant.

8. The information acquisition system according to claim 7, wherein the one or more processors and the one or more memories are further configured to acquire the information regarding the disinfection with the detection unit detecting the first information code, the second information code, and the third information code.

9. The information acquisition system according to claim 1, wherein the variable information code indicates the second information code after lapse of a certain time from the application of the disinfectant.

10. An article comprising:
a base; and
a color developing portion applied to the base,
wherein the color developing portion includes a visualization agent,
wherein a color developing state of the visualization agent changes upon application of a disinfectant,
wherein the color developing portion forms, on a surface of the base, a variable information code capable of being detected by a detection unit,
wherein, before application of a disinfectant, the variable information code is a first information code, and
wherein application of the disinfectant changes the color developing state of the visualization agent such that the variable information code changes to a second information code.

11. An information acquisition system of acquiring information regarding a chemical reaction, the information acquisition system comprising:
a detection unit configured to detect a variable information code in which a pattern before occurrence of the chemical reaction represents a first information code and a pattern after the occurrence of the chemical reaction represents a second information code different from the first information code;
one or more memories; and
one or more processors in communication with the detection unit and with the one or more memories, wherein the one or more processors and the one or more memories are configured to:
acquire the information regarding the chemical reaction with detection of at least the second information code by the detection unit.

12. An object comprising:
a base; and
a color developing portion applied to the base,
wherein the color developing portion includes a visualization agent,
wherein a color developing state of the visualization agent changes upon a chemical reaction,
wherein the color developing portion forms, on a surface of the base, a variable information code capable of being detected by a detection unit,
wherein, before an occurrence of the chemical reaction, the variable information code is a first information code, and
wherein the occurrence of the chemical reaction changes the color developing state of the visualization agent such that the variable information code changes to a second information code.

13. A method of acquiring information regarding disinfection, the method comprising:
detecting a variable information code in which a pattern before application of a disinfectant represents a first information code and a pattern after the application of the disinfectant represents a second information code different from the first information code; and
acquiring the information regarding the disinfection with detection of at least the second information code by the detection unit.
